(19) United States
(12) Plant Patent Application Publication
(10) Pub. No.: US 2022/0248587 P1
(43) Pub. Date: Aug. 4, 2022

(54) BLUEBERRY PLANT NAMED 'C12-069'

(50) ;

(71) Applicants: CostaExchange Pty Ltd, Ravenhall (AU); FLORIDA FOUNDATION SEED PRODUCERS, INC., Marianna, FL (US)

(72) Inventors: Gary Wright, Arrawarra Headland (AU); Paul M. Lyrene, Micanopy, FL (US)

(21) Appl. No.: 17/803,053

(22) Filed: Jan. 26, 2022

Related U.S. Application Data
(60) Provisional application No. 63/206,154, filed on Jan. 29, 2021.

(51) Int. Cl. A01H 6/36

(52) U.S. Cl. PLT/157

(57) ABSTRACT

The new blueberry plant variety ‘C12-069’ is provided. ‘C12-069’ is a commercial variety intended for the fresh market. The variety is produced from a cross of ‘Indigocrisp’ (seed parent, U.S. Plant Pat. No. 26,523) and ‘FL01-271’ (unpatented pollen parent).

### CROSS-REFERENCE TO RELATED APPLICATIONS

This application claims priority to U.S. Application No. 63/206,154 filed on 29 Jan. 2021, the contents of which are hereby incorporated in their entirety by reference.

Latin name of the family, genus, and species: Family—Ericaceae. Genus—Vaccinium. Species—corymbosum hybrid.

Variety denomination: The new blueberry plant claimed is of the variety denominated ‘C12-069’.

### BACKGROUND OF THE INVENTION

The new variety ‘C12-069’ was selected from a population of seedlings derived from crossing the blueberry varieties known as ‘Indigocrisp’ (seed parent) (U.S. Plant Pat. No. 26,523) and the variety known as ‘FL01-271’ (unpatented pollen parent). The cross was made in 2006 in Florida, USA and the seed was sown and grown in Corindi Beach, New South Wales, Australia. The new variety was selected in 2012 from among plants located on land at Corindi Beach and assigned the breeding code ‘C12-069’. Plants of ‘C12-069’ were propagated by cuttings for further evaluation and resulted to be uniform and stable. The new variety showed distinctive traits such as evergreen, with fruit of good flavor, large fruit size, and firmness.

### SUMMARY OF THE INVENTION

The new variety ‘C12-069’ was originated from a cross of ‘Indigocrisp’ (seed parent) (U.S. Plant Pat. No. 26,523) and the variety known as ‘FL01-271’ (unpatented pollen parent) in 2006 in Florida, USA.

The new blueberry variety resulted from seedlings produced in a controlled breeding programme. The cross was made in 2006 in Florida, USA and the seed was sown and grown in Corindi Beach, New South Wales, Australia.

The new variety was selected in 2012 from among plants located on land at Corindi Beach and has since been named ‘C12-069’. Since then plants of ‘C12-069’ were propagated by cuttings for further evaluation and confirmed to be uniform and stable. Asexual reproduction of the new variety ‘C12-069’ by cutting propagation since 2012 at Corindi Beach, New South Wales, Australia has demonstrated that the new variety reproduces true to type plants.

The new variety was selected in 2012 as a single plant within a population of seedlings resulting from controlled cross of Vaccinium varieties. The seedling population was planted in an experimental block in the field at Corindi Beach, New South Wales, Australia and the selection of the new variety took place in the same block. Selection criteria were a combination of early cropping time, between the very early to early cropping variety ‘Snowchaser’ and the early to mid season cropping variety ‘C99-042’. ‘C12-069 has estimated low chilling requirement between 150 to 300 hours, is non-deciduous type of plant (evergreen), very large fruit size, good fruit flavor, firm fruit and long shelf life. The new variety was subsequently evaluated for seven years at the commercial farm at Corindi Beach, New South Wales, Australia.

The following characteristics of the new variety have been repeatedly observed and can be used to distinguish ‘C12-069’ as a new and distinct variety of Vaccinium corymbosum hybrid:
1. Non-deciduous (Evergreen)
2. Early season crop
3. Very large fruit size (4.1 g average)
4. Low chilling requirement
5. Excellent firmness and good flavor
6. Long shelf life

The new blueberry variety ‘C12-069’ has maintained its distinguished characteristics throughout successive asexual propagation. The variety has been repeatedly asexually reproduced through softwood cuttings in New South Wales, Australia and the clones are phenotypically identical to the original plant.

### BRIEF DESCRIPTION OF THE DRAWINGS

The accompanying photographic illustration shows typical specimens in full color of the foliage and fruit of the new variety ‘C12-069’. The colors are as nearly true as is reasonably possible in a color representation of this type.

FIG. 1 is a photograph of the new variety ‘C12-069’ showing botanical parts of the plant (ripe fruit, green fruit, flowers and leaves).

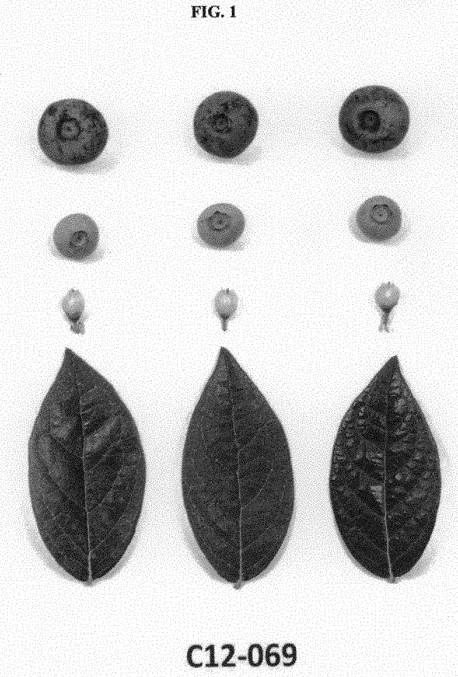

FIG. 2 is a photograph of the new variety ‘C12-069’, showing a typical fruit calyx of the variety.

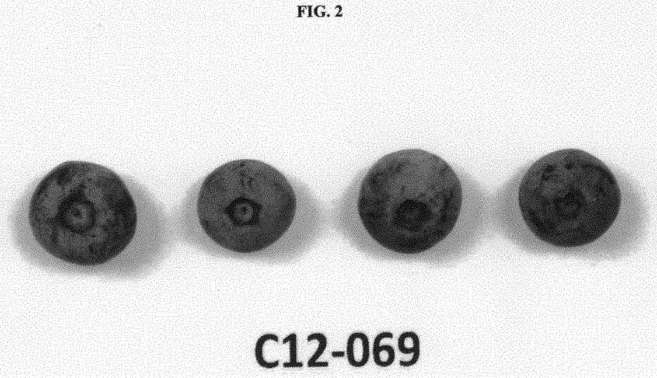

FIG. 3 is a photograph of ‘C12-069’ showing a typical pedicel scar of a fruit of the variety.

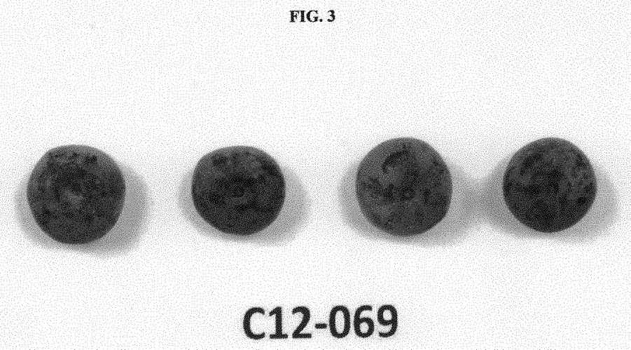

FIG. 4 shows the typical fruit cluster of the new variety ‘C12-069’.

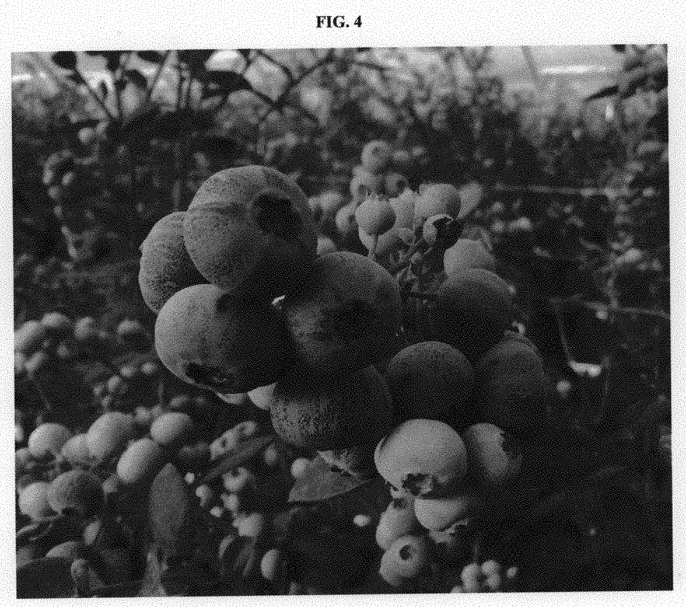

FIG. 5 shows a full row of the new variety ‘C12-069’ growing in pots and managed in substrate.

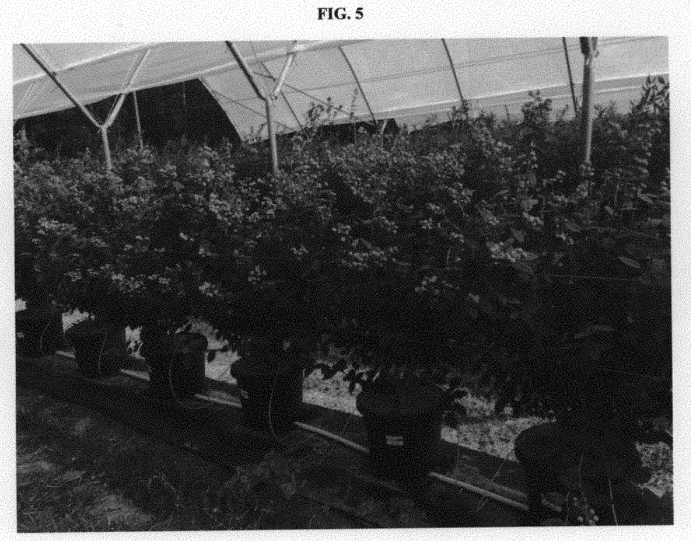

The colors in the photographs are as close as possible with the photographic and printing technology utilized. The color values cited in the detailed botanical description accurately describe the colors of the new blueberry variety.

### DETAILED BOTANICAL DESCRIPTION

The following detailed description sets forth the distinctive characteristics of ‘C12-069’. The data which defines these characteristics was collected from asexual reproductions of the original selection. Dimensions, sizes, colors, and other characteristics are approximations and averages set forth as accurately as possible. For all traits data was collected from 3 plant parts across 6 randomly selected plants. For the traits relating to fruits (e.g., fruit weight, firmness, brix, acidity) the data is an average across twenty fruits collected randomly. The plant history was taken on plants approximately 3 years of age, and the descriptions relate to plants grown in the field in Corindi Beach, New South Wales, 2456 Australia. Descriptions of fruit characteristics were made on fruit grown in Corindi Beach, New South Wales, 2456 Australia. Color designations are from the 2007 edition of the Royal Horticultural Society (“RHS”).

Classification:
a. Family—Ericaceae.
b. Genus—Vaccinium.
c. Species—corymbosum hybrid.
d. Common Name—blueberry.

Parentage: Female Parent—patented variety ‘Indigocrisp’. Male Parent—unpatented selection ‘FL01-271’.

Market Class: fresh market.

### PLANT

General:
   Parentage.—‘Indigocrisp’x FL01-271.
   Plant height.—1.44 m.
   Plant width.—0.92 m.
   Growth habit.—upright.
   Growth.—vigour is medium.
   Productivity.—medium.
   Cold hardiness.—low chill.
   Cold tolerance.—low.
   Chilling requirement.—low, estimated between 150 and 300 hours.
   Tolerance to disease.—none assessed.
   Leafing.—overall medium to high and plant retains leaves during the winter.
   Twigginess.—low.

### FOLIAGE

General:
   Leaf color (top side).—green group, similar to N137A.
   Leaf color (under side).—yellow-green, similar to 148D.
   Leaf arrangement.—alternate.
   Leaf shape.—elliptic.
   Leaf margins.—entire.
   Leaf venation.—reticulate.
   Leaf length.—average 64.7 mm (long to very long).
   Leaf width.—average 34.3 mm (broad).
   Leaf length/width ratio.—1.9.
   Leaf nectarines.—absent.
   Pubescence of upper side.—absent.
   Pubescence of lower side.—absent.
   Attitude.—horizontal.
Petioles:
   Length.—average 3.4 mm.
   Width.—average 1.7 mm.
   Colour.—similar to yellow-green group 145A.

### FLOWERS

General:
   Time of beginning to flower.—early season.
   Flower shape.—urceolate.
   Flower fragrance.—none perceptible.
Corolla:
   Color.—white group, similar to NN155B.
   Length.—medium, averaging 9.6 mm, similar to that of ‘Snowchaser’ and that of ‘C99-042’.
   Width.—7 mm.
   Aperture width.—3.6 mm.
   Anthocyanin coloration of corolla.—absent or very weak.
   Corolla ridges.—present.
   Protrusion of stigma.—absent.
Inflorescence:
   Surface texture of peduncle.—medium.
   Color of peduncle.—the base colour is similar to yellow-green group 145A, and the overall colour is similar to greyed-red group 182A.
   Length of pedicel.—average 14.6 mm.
   Surface texture of pedicel.—smooth.
   Color of pedicel.—yellow green group, similar to 145B and 145C.
   Number of flowers per cluster.—average 8.
   Flower cluster density.—dense.
Stamen:
   Length.—average 7.7 mm.
   Number per flower.—9.
   Filament color.—similar to yellow-green group 145D.
   Style.—Length — average 9.15 mm.
   Color.—similar to yellow-green group 145A.
Pistil:
   Length.—average 12.03 mm.
Anther:
   Length.—average 4.4 mm.
   Number.—9.
   Color.—similar to greyed-orange group 165A.
Pollen:
   Abundance.—little.
   Color.—similar to yellow group 11C.
   Self compatibility.—yes.

### FRUIT

General:
   Cluster density.—dense, ranging 6-9 berries per cluster.
   Unripe fruit color.—similar to green group 144A.
   Ripe berry color.—blue group 103A.
   Berry surface wax abundance.—medium.
   Berry weight.—very large, on average 4.1 g.
   Berry height from calyx to scar.—14.3 mm.
   Berry diameter.—19.6 mm.
   Berry shape.—oblate.
   Fruit stem scar.—dry.
   Sweetness when ripe.—medium (12 Brix).
   Acidity when ripe.—medium (0.6%).
   Storage quality.—long shelf life (36 days).
   Suitability for mechanical harvesting.—not tested.
   Self-fruitfulness.—yes, with a proportion of the fruit parthenocarpic.
   Uses.—fruit to be hand harvested for tresh market.

### SEED

General:
   Seed abundance in fruit.—low, on average 5 seeds per fruit.
   Seed color.—similar to greyed orange 165A.
   Seed length.—average 2.14 mm.

### COMPARISON WITH COMMERCIAL CULTIVARS

Table 1 below provide comparison between ‘C12-069’ and several commercial cultivars:

Comparison of ‘C12-069’ with similar cultivars

| Characteristic | C12-069 | C99-042 | Snowchaser |
|---|---|---|---|
| Plant vigour | Medium | Weak to medium | Medium |
| Plant growth habit | Upright | Semi-upright to | Semi-upright |
| | | intermediate | |
| One year old shoot | Medium | Very short to short | Short |
| length of internodes | | | |
| Leaf length | Long to very long | short | Long |
| Leaf length (mm) | 64.7 ± 2.6 | 53.10 ± 0.8 | 61.50 ± 0.9 |
| Leaf width | Broad | Very narrow to narrow | Broad |
| Leaf width (mm) | 34.3 ± 1.8 | 22.9 ± 1.3 | 34.3 ± 1.5 |
| Flower size of | Medium | Medium | Medium |
| corolla | | | |
| Flower corolla | 9.6 ± 0.3 | 9.7 ± 0.3 | 9.5 ± 0.3 |
| length (mm) | | | |
| Fruit cluster density | Dense | Sparse | Medium |
| Fruit size | Very large | Small to medium | Small to medium |
| Fruit weight (g) | 4.1 ± 0.2 | 1.9 ± 0.39 | 1.7 ± 0.21 |
| Fruit diameter (mm) | 19.6 ± 1.7 | 15.6 ± 0.96 | 15.10 ± 0.97 |
| Fruit depth of calyx | Shallow | Medium | Shallow |
| basin | | | |
| Fruit intensity of | Medium | Weak to medium | Weak to medium |
| bloom | | | |
| Fruit firmness | Very firm | Firm | Soft |
| Soluble solid | 12 | 13.1 | 14 |
| content (%) | | | |
| Titratable acidity | 0.6 | 0.4 | 0.7 |
| (%) | | | |
| Time of vegetative | Early | Early | Early |
| bud burst | | | |
| Time of beginning | Early | Early to medium | Very early to early |
| of flowering | | | |
| Time of beginning | Early | Early to medium | Very early to early |
| of fruit ripening | | | |

1. A new and distinct variety of blueberry plant named ‘C12-069’, substantially as illustrated and described herein.
